(19) United States
(12) Patent Application Publication
(10) Pub. No.: US 2025/0166928 A1
(43) Pub. Date: May 22, 2025

(54) SOLID ELECTROLYTIC CAPACITOR AND METHOD FOR PRODUCING SOLID ELECTROLYTIC CAPACITOR

(71) Applicant: Panasonic Intellectual Property Management Co., Ltd., Kadoma-shi, Osaka (JP)

(72) Inventors: Kentaro Koyanagi, Saga Ken (JP); Tomoya Takasaki, Osaka Fu (JP); Nobuyuki Yamaguchi, Saga Ken (JP); Kohei Goto, Osaka Fu (JP)

(73) Assignee: Panasonic Intellectual Property Management Co., Ltd., Kadoma-shi, Osaka (JP)

(21) Appl. No.: 18/832,649

(22) PCT Filed: Jan. 20, 2023
(86) PCT No.: PCT/JP2023/001605

§ 371 Date: Jul. 24, 2024

(30) Foreign Application Priority Data
Jan. 28, 2022 (JP) 2022-011884

(51) Int. Cl. H01G 9/025; C09D 5/24; C09D 141/00; C09D 165/00; H01G 9/00; H01G 9/15

(52) U.S. Cl. CPC H01G 9/025; C09D 5/24; C09D 141/00; C09D 165/00; H01G 9/0036; H01G 9/15

(57) ABSTRACT

A solid electrolytic capacitor that includes an anode body having a porous part on surface thereof and a manufacturing method thereof including a step (i) of forming a first solid electrolyte layer on a dielectric layer and a step (ii) of forming a second solid electrolyte layer on the first solid electrolyte layer. The first and second solid electrolyte layers contain first and second conductive polymers, respectively. The step (i) includes a step (i-a) of supplying a reaction solution containing a monomer and a silane compound to the surface of the dielectric layer, and a step (i-b) of forming the first solid electrolyte layer by polymerizing the monomer in the supplied reaction solution to form the first conductive polymer. The monomer contains a compound represented by the following formula (I) (in the formula (I), R represents an alkyl group whose carbon number is within a range of 1 to 10).

### TECHNICAL FIELD

The present disclosure relates to a solid electrolytic capacitor and a manufacturing method for the same.

### BACKGROUND ART

As a method for forming a conductive polymer layer in a solid electrolytic capacitor, there has been proposed a method in which a monomer is polymerized on the surface of a dielectric layer.

PTL 1 (Japanese Patent No. 5093915) discloses “a solid electrolytic capacitor formed by polymerizing a monomer mixture of 2,3-dihydro-thieno[3,4-b][1,4]dioxine and 2-alkyl-2,3-dihydro-thieno[3,4-b][1,4]dioxine at a molar ratio of 0.05:1 to 1:0.1 on a capacitor element that has an anode made of a porous valve metal selected from tantalum, niobium, and aluminum and a dielectric layer made of an oxide film of the valve metal in the presence of an organic sulfonic acid to form a conductive polymer layer containing the organic sulfonic acid as a dopant, the conductive polymer serving as a solid electrolyte, in which the capacitor element does not use a separator”.

PTL 2 (Japanese Patent No. 6695023) discloses “a method for manufacturing an electrolytic capacitor, the method including: a first step of preparing an anode member having a dielectric layer; a second step of impregnating the anode member with a monomer, an oxidizing agent, a silane compound, and a solvent; and a third step of forming a solid electrolyte layer on a surface of the dielectric layer, the solid electrolyte layer including a conductive polymer including a polymer of the monomer and a silicon-containing component derived from the silane compound, in which the monomer includes a compound represented by a predetermined formula where R in the predetermined formula is an alkyl group whose carbon number is 2 to 4”. The structure of the predetermined formula is the same as the structure of formula (I) described later.

### CITATION LIST

### Patent Literatures

PTL 1: Japanese Patent No. 5093915
PTL 2: Japanese Patent No. 6695023

### SUMMARY OF INVENTION

### Technical Problem

Currently, there is demand for improvement in characteristics (for example, reduction in leakage current, improvement in voltage resistance, and the like) and reliability of solid electrolytic capacitors. In this situation, an object of the present disclosure is to provide a solid electrolytic capacitor with improved characteristics.

### Solution to Problem

One aspect of the present disclosure relates to a manufacturing method for a solid electrolytic capacitor. The manufacturing method is a manufacturing method for a solid electrolytic capacitor that includes an anode body having a porous part on a surface thereof and a dielectric layer formed on at least a portion of the surface of the porous part. The manufacturing method includes a step (i) of forming a first solid electrolyte layer that covers at least a portion of the dielectric layer, and a step (ii) of forming a second solid electrolyte layer that covers at least a portion of the first solid electrolyte layer. The first solid electrolyte layer contains a first conductive polymer. The second solid electrolyte layer contains a second conductive polymer. The step (i) includes a step (i-a) of supplying a reaction solution containing a monomer and a silane compound to a surface of the dielectric layer, and a step (i-b) of forming the first solid electrolyte layer by polymerizing the monomer in the supplied reaction solution to form the first conductive polymer. The monomer contains a compound represented by the following formula (I):

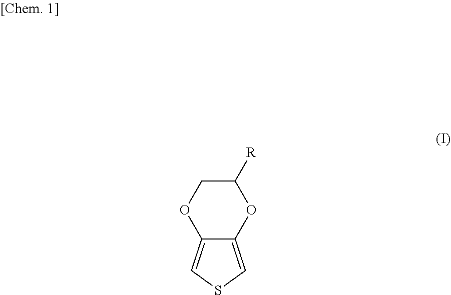

(In the formula (I), R represents an alkyl group whose carbon number is within a range of 1 to 10).

Another aspect of the present disclosure relates to a solid electrolytic capacitor. The solid electrolytic capacitor includes an anode body that has a porous part on a surface thereof, a dielectric layer that is formed on at least a surface of a portion of the porous part, a first solid electrolyte layer that covers at least a portion of the dielectric layer, and a second solid electrolyte layer that covers at least a portion of the first solid electrolyte layer. The first solid electrolyte layer contains a first conductive polymer and a silicon-containing component. The second solid electrolyte layer contains a second conductive polymer. The first conductive polymer is a polymer of a monomer. The monomer contains a compound represented by the above formula (I) (in the formula (I). R represents an alkyl group whose carbon number is within a range of 1 to 10).

### Advantageous Effects of Invention

According to the present disclosure, it is possible to obtain a solid electrolytic capacitor with excellent characteristics.

Novel features of the present invention are set forth in the appended claims. The present invention, both in terms of structure and content, will be better understood together with other objects and features of the present invention, from the following detailed description with reference to the drawings.

### BRIEF DESCRIPTION OF DRAWINGS

FIG. 1 is a schematic cross-sectional view of a solid electrolytic capacitor according to a first embodiment.

FIG. 2 is a schematic cross-sectional view of a portion of the solid electrolytic capacitor illustrated in FIG. 1.

### DESCRIPTION OF EMBODIMENTS

Hereinafter, embodiments according to the present invention will be described taking examples, but the present invention is not limited to the examples described below. In the following description, specific numerical values and materials are given as examples in some cases, but other numerical values and other materials may be applied as long as the invention according to the present disclosure can be implemented. In the specification, the expression “numerical value A to numerical value B” includes numerical value A and numerical value B, and can be read as “numerical value A or more and numerical value B or less”. In the following description, if lower and upper limits of numerical values related to specific physical properties or conditions are given as examples, any of the lower limits given as examples and any of the upper limits given as examples can be combined as desired as long as the lower limit is not greater than or equal to the upper limit.

### (Manufacturing Method for Solid Electrolytic Capacitor)

A manufacturing method according to the present embodiment is a manufacturing method for a solid electrolytic capacitor that includes an anode body having a porous part on its surface and a dielectric layer formed on at least a portion of the surface of the porous part. Hereinafter, the manufacturing method will also be referred to as “manufacturing method (M)”. Hereinafter, the solid electrolytic capacitor will also be referred to as “electrolytic capacitor” or “capacitor”.

The manufacturing method (M) includes steps (i) and (ii) in this order. As described later, according to the manufacturing method (M) including the steps (i) and (ii), it is possible to form a solid electrolyte layer that includes a first solid electrolyte layer and a second solid electrolyte layer. The first solid electrolyte layer contains a first conductive polymer that contains a constitutional unit derived from an alkyl EDOT described later, and a silicon-containing component. Using the first conductive polymer that contains the constitutional unit derived from the alkyl EDOT reduces the leakage current and increases the withstand voltage. This is considered to be because the use of the alkyl EDOT changes the orientation of the conductive polymer in the first solid electrolyte layer, making it difficult for the current to flow in the inter-electrode direction.

Unlike in the case of forming the electrolyte layer using a dispersion liquid of a conductive polymer, the electrolyte layer can be formed more uniformly on the surface of the intricate porous part in situ through polymerization. Therefore, it is possible to reduce the ESR and improve the capacitance. However, if the electrolyte layer is only formed through polymerization, the electrolyte layer tends to be thin, and therefore the leakage current is likely to increase and the withstand voltage is likely to decrease. In the manufacturing method (M), a silane compound is added to a reaction solution that forms the first solid electrolyte layer, and the second solid electrolyte layer is formed. This makes it possible to reduce the leakage current and increase the withstand voltage.

In addition, combining the formation of the first conductive polymer using alkyl EDOT with the use of a silane compound makes it possible to improve the withstand voltage while relatively suppressing the increase in the ESR.

The step (i) and step (ii) will be described below.

### (Step (i))

The step (i) is a step of forming the first solid electrolyte layer that covers at least a portion of the dielectric layer. The first solid electrolyte layer contains the first conductive polymer. The step (i) includes a step (i-a) of supplying a reaction solution containing a monomer and a silane compound to the surface of the dielectric layer, and a step (i-b) of forming the first solid electrolyte layer by polymerizing the monomer in the supplied reaction solution to form a first conductive polymer. The monomer (at least one type of monomer) contains a compound represented by the following formula (I). Hereinafter, the compound will also be referred to as “alkyl EDOT”.

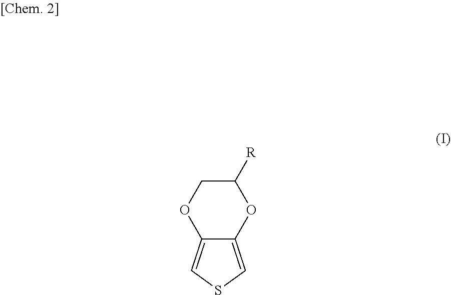

(In the formula (I), R represents an alkyl group whose carbon number is within a range of 1 to 10).

The carbon number of the alkyl group represented by R may be within a range of 1 to 6 (for example, within a range of 1 to 4 or a range of 2 to 4), may be 2 or 3, or may be 3 or 4. The monomer may contain only one type of an alkyl EDOT, or may contain a plurality of types of alkyl EDOTs different in R. When the carbon number of the alkyl group represented by R is 3 or more, the alkyl group may be linear or branched.

The monomer may further contain 3,4-ethylenedioxythiophene represented by the following formula. Hereinafter, 3,4-ethylenedioxythiophene will also be referred to as “EDOT”. The first conductive polymer may be a copolymer of alkyl EDOT and EDOT. The monomer may contain other compounds.

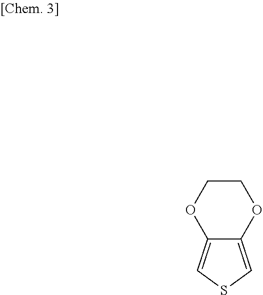

The reaction solution may satisfy the following condition (1):

(1) The proportion of the alkyl EDOT in all the monomers in the reaction solution is 25 mol % or more, or 50 mol % or more, and 90 mol % or less, or 75 mol % or less.

The reaction solution may satisfy the following condition (2) and/or (3). For example, the reaction solution may satisfy both the conditions (2) and (3). In these cases, the reaction solution may further satisfy the condition (1).

(2) The total proportion of the alkyl EDOT and EDOT in all the monomers in the reaction solution is within a range of 80 to 100 mol %, 90 to 100 mol %, or 95 to 100 mol % (for example, 100 mol %).

(3) In the reaction solution, a value Y of (number of moles of the alkyl EDOT)/(number of moles of the alkyl EDOT+number of moles of EDOT) is 0.10 or more, 0.25 or more, or 0.50 or more, and is 1.0 or less, 0.75 or less, or 0.50 or less. The value Y may be within a range of 0.25 to 1.0, or within a range of 0.25 to 0.75.

The silane compound may be a silane coupling agent. In the reaction solution, a value X of (mass of the silane compound)/(sum total of mass of the monomer, mass of the oxidizing agent, and mass of the liquid medium of the reaction solution) may be 0.05 or more, 0.10 or more, 0.15 or more, or 0.20 or more, and may be 0.40 or less, 0.30 or less, or 0.20 or less. The value X may be within a range of 0.05 to 0.40, within a range of 0.05 to 0.30, or within a range of 0.10 to 0.30. Setting the value X in these ranges makes it possible to obtain a capacitor with low ESR, high withstand voltage, and low leakage current.

In the manufacturing method (M), the value Y may be within any of the above ranges, and the value X may be within any of the above ranges. For example, the value Y may be within the range of 0.25 to 0.75, and the value X may be within the range of 0.05 to 0.30.

The silane coupling agent is a silane compound having a hydrolyzable group (for example, an alkoxy group). The silane compound preferably has an epoxy group or an acrylic group since it is advantageous in reducing the ESR and increasing the capacitance. As the silane coupling agent, only one type of silane coupling agent may be used, or two or more types of silane coupling agents may be used in combination.

Examples of the silane compound (silane coupling agent) having an epoxy group include 2-(3,4-epoxycyclohexyl)ethyltrimethoxysilane, 3-glycidoxypropyltrimethoxysilane (γ-glycidoxypropyltrimethoxysilane), 3-glycidoxypropylmethyldiethoxysilane, 3-glycidoxypropyltriethoxysilane, and the like.

Examples of the silane compound (silane coupling agent) having an acrylic group include 3-methacryloxypropylmethyldimethoxysilane, 3-methacryloxypropyltrimethoxysilane, 3-methacryloxypropylmethyldiethoxysilane, 3-methacryloxypropyltriethoxysilane, 3-acryloxypropyltrimethoxysilane (γ-acryloxypropyltrimethoxysilane), and the like.

Examples of other silane coupling agents include vinyltrichlorosilane, vinyltrimethoxysilane, vinyltriethoxysilane, p-styryltrimethoxysilane, N-2-(aminoethyl)-3-aminopropylmethyldimethoxysilane, N-2-(aminoethyl)-3-aminopropyltriethoxysilane, N-2-(aminoethyl)-3-aminopropyltrimethoxysilane, 3-aminopropyltrimethoxysilane, 3-aminopropyltriethoxysilane, 3-triethoxysilyl-N-(1,3-dimethyl-butylidene) propylamine, N-phenyl-3-aminopropyltrimethoxysilane, N-(vinylbenzyl)-2-aminoethyl-3-aminopropyltrimethoxysilane hydrochloride, 3-ureidopropyltriethoxysilane, 3-chloropropyltrimethoxysilane, 3-mercaptopropylmethyldimethoxysilane, 3-mercaptopropyltrimethoxysilane, bis(triethoxysilylpropyl) tetrasulfide, 3-isocyanatopropyltriethoxysilane, and the like.

There is no particular limitation on the liquid medium (solvent) of the reaction solution, and any liquid medium that can carry out polymerization without problems may be used. The liquid medium may be water, a mixture of water and a nonaqueous solvent, or a nonaqueous solvent. Examples of nonaqueous solvents include organic solvents and ionic liquids. Specifically, examples of nonaqueous solvents include alcohols such as methanol, ethanol, propanol, butanol, ethylene glycol, and propylene glycol, formaldehyde, amides such as N-methylacetamide, N,N-dimethylformamide, and N-methyl-2-pyrrolidone, esters such as methyl acetate, ethers such as 1,4-dioxane, ketones such as methyl ethyl ketone, and the like.

The polymerization of the monomer may be chemical polymerization. In the case of carrying out chemical polymerization, the reaction solution contains an oxidizing agent. As the oxidizing agent, an oxidizing agent capable of polymerizing a monomer can be used, and a known oxidizing agent may be used. Examples of the oxidizing agent include sulfuric acid, hydrogen peroxide, organic sulfonic acid metal salts, and the like. Examples of the metal ions of the organic sulfonic acid metal salt include iron (II), copper (II), chromium (VI), cerium (IV), manganese (VII), zinc (II), and the like.

As the organic sulfonic acid metal salt, an aromatic sulfonic acid metal salt is preferable. For example, a naphthalene sulfonic acid metal salt, a tetralin sulfonic acid metal salt, an alkylbenzene sulfonic acid metal salt, or an alkoxybenzene sulfonic acid metal salt can be used. Since an aromatic sulfonic acid metal salt has a function as a dopant in addition to a function as an oxidizing agent, it is not necessary to use a separate dopant. Furthermore, an aromatic sulfonic acid metal salt has an excellent function as a dopant, and therefore a high-quality conductive polymer can be formed. In particular, it is preferable to use p-toluenesulfonate iron (III), which produces a conductive polymer with excellent conductivity and heat resistance.

The reaction solution may contain other components as necessary. For example, the reaction solution may contain a dopant. Examples of the dopant added to the reaction solution include non-polymer dopants. By using a non-polymer dopant, the reaction solution can easily permeate into the porous part.

The step (i-a) can be performed by bringing the dielectric layer on the surface of the anode body into contact with the reaction solution. For example, the reaction solution may be supplied to the surface of the dielectric layer by immersing the anode body in the reaction solution.

The step (i-b) can be performed by causing a polymerization reaction in a state where the dielectric layer on the surface of the anode body is in contact with the reaction solution. The polymerization reaction may be caused by leaving the reaction solution to stand, or performing treatment for promoting the reaction. For example, heating treatment may be carried out. The first solid electrolyte layer (first conductive polymer layer) is formed by the step (i-b).

(Step (ii))

The step (ii) is a step of forming the second solid electrolyte layer that covers at least a portion of the first solid electrolyte layer. The second solid electrolyte layer contains a second conductive polymer. The second solid electrolyte layer may contain the second conductive polymer and a dopant. Examples of these will be described later. The second conductive polymer may be the same as or different from the first conductive polymer. In a preferred example, the first conductive polymer is a copolymer of alkyl EDOT and EDOT, and the second conductive polymer is poly(3,4-ethylenedioxythiophene) (PEDOT).

The step (ii) may be performed using a known method for forming a solid electrolyte layer in a solid electrolytic capacitor. The step (ii) may include steps (ii-a) and (ii-b) in this order. The step (ii-a) is a step of applying a dispersion liquid that contains the second conductive polymer and a dispersion medium to the first solid electrolyte layer. The step (ii-b) is a step of forming the second solid electrolyte layer by removing at least a portion of the dispersion medium from the applied dispersion liquid.

The step (ii-a) can be performed by bringing the first solid electrolyte layer formed on the dielectric layer into contact with the dispersion liquid. For example, the first solid electrolyte layer may be immersed in the dispersion liquid, or the dispersion liquid may be applied to the first solid electrolyte layer. In the step (ii-b), there is no limitation on the method for removing at least a portion of the dispersion medium from the dispersion liquid applied to the first solid electrolyte layer, and a known method may be used. For example, the dispersion medium may be removed through heating and/or pressure reduction. The formation step including the step (ii-a) and the step (ii-b) may be repeated a plurality of times. Repeating the formation step makes it possible to thicken the second solid electrolyte layer.

The dispersion medium in the dispersion liquid is not limited, and may be water or a mixture of water and an organic solvent. The dispersion liquid may contain a dopant. The second conductive polymer may be dispersed in the dispersion medium in the form of particles.

There is no particular limitation on the concentration of the second conductive polymer in the dispersion liquid. The concentration may be within a range of 0.5 to 5.0 mass % (for example, 1.0 to 3.0 mass %).

In this manner, the solid electrolyte layer is formed including the first solid electrolyte layer and the second solid electrolyte layer. Thereafter, a step required for manufacturing a solid electrolytic capacitor is performed. The step is not particularly limited, and a known step for manufacturing a solid electrolytic capacitor may be performed. For example, first, a cathode extraction layer is formed so as to cover at least a portion of the solid electrolyte layer (more specifically, the second solid electrolyte layer). In this manner, a capacitor element is obtained. Next, lead terminals are connected to the capacitor element. For example, an anode lead terminal is electrically connected to the anode body or an anode wire, and a cathode lead terminal is electrically connected to the cathode extraction layer. Next, part of the lead terminals and the capacitor element are enclosed in an exterior body. In this manner, a solid electrolytic capacitor is obtained.

The anode body may be a tantalum sintered body. The tantalum sintered body is preferable in that it is porous, has a large surface area relative to its volume, and can increase the electrostatic capacitance. The second conductive polymer may contain poly(3,4-ethylenedioxythiophene) (hereinafter, also referred to as “PEDOT”), or may be poly(3,4-ethylenedioxythiophene). The PEDOT is preferable in that it is capable of forming a favorable solid electrolyte layer.

(Solid electrolytic capacitor)

The solid electrolytic capacitor according to the present embodiment will also be referred to as “capacitor (C)” below. The capacitor (C) can be manufactured using the manufacturing method (M). Thus, the matter describing the manufacturing method (M) can be applied to the capacitor (C), and thus redundant description will be omitted. Furthermore, the matter describing the capacitor (C) can be applied to the manufacturing method (M). However, the capacitor (C) may be formed using a method other than the manufacturing method (M).

The capacitor (C) includes an anode body that has a porous part on its surface, a dielectric layer that is formed on at least the surface of a portion of the porous part, a first solid electrolyte layer that covers at least a portion of the dielectric layer, and a second solid electrolyte layer that covers at least a portion of the first solid electrolyte layer. The first solid electrolyte layer contains a first conductive polymer and a silicon-containing component. The second solid electrolyte layer contains a second conductive polymer. The first conductive polymer is a polymer of a monomer. The monomer contains a compound (alkyl EDOT) represented by the above formula (I).

As described above, the monomer may contain a compound other than the alkyl EDOT. That is, the first conductive polymer may contain a constitutional unit derived from the alkyl EDOT and a constitutional unit derived from another compound. Examples of the other compound include 3,4-ethylenedioxythiophene and the like.

The first solid electrolyte layer can be formed by the above-described step (i). The silicon-containing component contained in the first solid electrolyte layer may be derived from the silane coupling agent used in the step (i). The silicon-containing component may be a component formed by subjecting the silane coupling agent to hydrolytic condensation.

The second solid electrolyte layer may be formed by the above-described step (ii). The second conductive polymer may contain poly(3,4-ethylenedioxythiophene) (PEDOT).

Examples of the solid electrolytic capacitor manufactured using the manufacturing method (M) and the structure and components of the capacitor (C) will be described below. The solid electrolytic capacitor of the example described below includes a capacitor element, an exterior body, an anode lead terminal, and a cathode lead terminal. The solid electrolytic capacitor manufactured using the manufacturing method (M) and the structure and components of the capacitor (C) are not limited to the following examples.

### (Capacitor Element)

The capacitor element includes an anode part, a dielectric layer, a solid electrolyte layer, and a cathode extraction layer.

The anode part includes an anode body and may further include an anode wire. The anode body may be a porous sintered body or may be a metal foil with a porous surface. The dielectric layer is formed on at least a portion of the surface of the anode body. The solid electrolyte layer is disposed between the dielectric layer formed on the surface of the anode body and the cathode extraction layer. There are no particular limitations on the components other than the solid electrolyte layer, and components used in known solid electrolytic capacitors may be applied. Examples of these components will be described below.

### (Anode Body)

The material of the anode body may be a valve metal. Examples of the valve metal include titanium (Ti), tantalum (Ta), niobium (Nb), aluminum (Al), and alloys containing these metals. The anode body having a porous part on its surface may be formed by sintering particles of the material (for example, particles of a valve metal). Alternatively, the anode body having a porous part on its surface may be formed by etching the surface of a metal foil. The dielectric layer formed on the surface of the anode body may be formed by subjecting the surface of the anode body to chemical conversion treatment. There is no limitation on the method of chemical conversion treatment, and a known method of chemical conversion treatment may be applied.

Capacitors with a sintered body (for example, a tantalum sintered body) as an anode body usually do not include a separator. In a capacitor that does not include a separator, the gap between the anode body and the cathode extraction layer is narrow, and therefore it is particularly important to suppress leakage current. Therefore, the manufacturing method (M) and the configuration of the capacitor (C) are particularly preferable for a capacitor that does not include a separator. If the capacitor according to the present disclosure includes a separator, there is no limitation on the separator, and a known separator may be used.

### (Anode Wire)

When the anode body is a sintered body, the anode part may include an anode wire. The anode wire may be a wire made of a metal. Examples of materials for the anode wire include the valve metals described above, copper, and the like. A portion of the anode wire is embedded in the anode body, and the remaining portion protrudes from the end face of the anode body.

### (Solid Electrolyte Layer)

The first solid electrolyte layer is formed using the above-described method. Examples of the second conductive polymer contained in the second solid electrolyte layer include polypyrrole, polythiophene, polyaniline, derivatives thereof, and the like. These may be used alone or in combination. The second conductive polymer may be a copolymer of two or more types of monomers. The derivative of a conductive polymer refers to a polymer having a conductive polymer as a basic skeleton. An example of a derivative of polythiophene includes poly(3,4-ethylenedioxythiophene).

It is preferable that a dopant is added to the conductive polymer. The dopant can be selected depending on the conductive polymer, and a known dopant may be used. Examples of the dopant include naphthalenesulfonic acid, p-toluenesulfonic acid, polystyrenesulfonic acid, salts thereof, and the like. A preferable example of the second solid electrolyte layer is formed by using poly(3,4-ethylenedioxythiophene) (PEDOT) doped with polystyrenesulfonic acid (PSS).

### (Cathode Extraction Layer)

The cathode extraction layer is a conductive layer and is disposed so as to cover at least a portion of the solid electrolyte layer (the second solid electrolyte layer). The cathode extraction layer may include a carbon layer formed on the electrolyte layer and a metal paste layer formed on the carbon layer. The carbon layer may be made of a conductive carbon material such as graphite and a resin. The metal paste layer may be made of metal particles (for example, silver particles) and a resin, and may be made of a known silver paste, for example.

### (Cathode Lead Terminal and Anode Lead Terminal)

The lead terminals (cathode lead terminal and anode lead terminal) are not particularly limited, and known lead terminals may be used. A portion of the cathode lead terminal is electrically connected to the cathode extraction layer. For example, the portion may be connected to the cathode extraction layer via a conductive layer (for example, a silver paste layer) or the like.

### (Exterior Body)

The exterior body is not limited, and a known exterior body may be used. The exterior body may be constituted of at least one selected from the group consisting of a resin composition, a film, and a case. An example of the exterior body is arranged around the capacitor element such that the capacitor element is not exposed on the surface of the electrolytic capacitor. Furthermore, the example of the exterior body is arranged so as to cover a portion of an anode lead frame and a portion of a cathode lead frame. The resin composition used as the exterior body may contain a resin (insulating resin) and an insulating filler.

Hereinafter, an example of the manufacturing method (M) and the capacitor (C) will be specifically described with reference to the drawings. The above description can be applied to the example described below. The example described below can be modified based on the above description. The matter described below may be applied to the above embodiment. In the embodiments described below, components that are not essential to the manufacturing method and electrolytic capacitor according to the present disclosure may be omitted. In order to facilitate understanding, the drawings referred to below may show shapes different from the actual shapes.

### First Embodiment

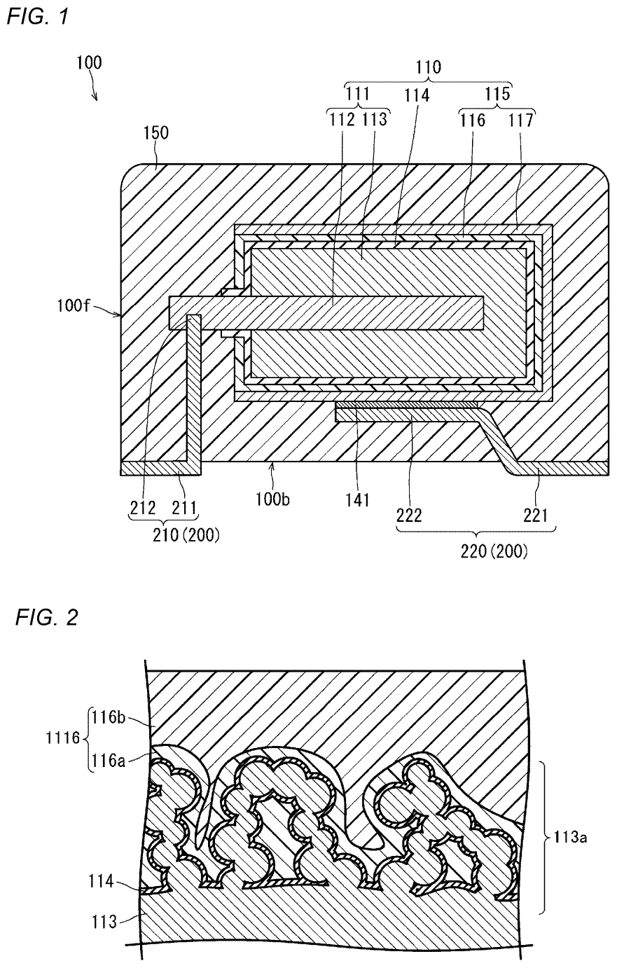

FIG. 1 is a schematic cross-sectional view of an example of the capacitor (C) according to a first embodiment. A capacitor (solid electrolytic capacitor) 100 shown in FIG. 1 includes a capacitor element 110, an exterior body 150, an anode lead terminal 210, and a cathode lead terminal 220. The exterior body 150 is disposed so as to cover a portion of the anode lead terminal 210, a portion of the cathode lead terminal 220, and the capacitor element 110.

The capacitor element 110 includes an anode part 111, a dielectric layer 114, and a cathode part 115. The anode part 111 includes an anode body 113 and an anode wire 112. The anode body 113 is a porous sintered body in the shape of a rectangular parallelepiped, and has a dielectric layer 114 formed on its surface. A portion of the anode wire 112 protrudes from one end face of the anode body 113 toward a front surface 100f of the electrolytic capacitor 100. The other portion of the anode wire 112 is embedded in the anode body 113.

The cathode part 115 includes a solid electrolyte layer 116 that is disposed so as to cover at least a portion of the dielectric layer 114, and a cathode extraction layer 117 that is formed on the solid electrolyte layer 116. The cathode extraction layer 117 includes a carbon layer formed on the solid electrolyte layer 116, and a metal particle layer formed on the carbon layer, for example. The metal particle layer is a metal paste layer (for example, a silver paste layer) formed using a metal paste, for example.

The anode lead terminal 210 includes an anode terminal part 211 and a wire connection part 212. The anode terminal part 211 is exposed on a bottom surface 100b of the electrolytic capacitor 100 (see FIG. 2). The wire connection part 212 is connected to the anode wire 112. The cathode lead terminal 220 includes a cathode terminal part 221 and a connection part 222. The cathode terminal part 221 is exposed on the bottom surface 100b of the electrolytic capacitor 100. The connection part 222 is electrically connected to the cathode extraction layer 117 (cathode part 115) via the conductive layer 141.

FIG. 2 is a schematic partial enlarged view of the anode body 113, the dielectric layer 114, and the solid electrolyte layer 116. Referring to FIG. 2, the anode body 113 has a porous part 113a at least on its surface. The dielectric layer 114 is formed on the surface of the porous part 113a. The solid electrolyte layer 116 includes a first solid electrolyte layer 116a and a second solid electrolyte layer 116b. The first solid electrolyte layer 116a is formed on the dielectric layer 114. The second solid electrolyte layer 116b is formed on the first solid electrolyte layer 116a. That is, the first solid electrolyte layer 116a and the second solid electrolyte layer 116b are laminated in this order on the dielectric layer 114.

The solid electrolyte layer 116 of the capacitor 100 is formed using the method described above. Specifically, the first solid electrolyte layer 116a is formed in the step (1), and the second solid electrolyte layer is formed in the step (ii). The first solid electrolyte layer 116a contains a first conductive polymer and a silicon-containing component. The second solid electrolyte layer 116b contains a second conductive polymer.

### EXAMPLES

The solid electrolytic capacitor and the manufacturing method thereof according to the present disclosure will be described in more detail with reference to examples. In the following examples, a plurality of solid electrolytic capacitors were produced and evaluated.

### (Capacitor A1)

A capacitor A1 was produced using the following procedure. First, a tantalum sintered body (porous body) in which a portion of an anode wire was embedded was prepared as an anode body. The surface of this tantalum sintered body was anodized to form a dielectric layer containing tantalum oxide on the surface of the anode body.

Next, a first solid electrolyte layer was formed on the surface of the dielectric layer through chemical polymerization. First, a reaction solution was prepared. The reaction solution was prepared by adding and mixing ferric p-toluenesulfonate (oxidizer), 3-acryloxypropyltrimethoxysilane (silane compound), and a monomer to ethanol (liquid medium). As the monomers, an alkyl EDOT in which R was a butyl group and 3,4-ethylenedioxythiophene (EDOT) were used. The reaction solution was prepared such that the values X and Y were as shown in Table 1. As described above, the value X is the value of (mass of the silane compound)/(total of the mass of the monomers, the mass of the oxidizer, and the mass of the liquid medium of the reaction solution). The value Y is the value of (number of moles of alkyl EDOT)/(number of moles of alkyl EDOT+number of moles of EDOT).

Next, the tantalum sintered body was immersed in the reaction solution for about 3 to 10 seconds. After it was pulled out of the reaction solution, the tantalum sintered body was heated at 210° C. for 3 minutes to polymerize the monomers. In this manner, a first solid electrolyte layer containing a first conductive polymer and a silicon-containing component was formed.

Next, a second solid electrolyte layer was formed using a dispersion liquid of a second conductive polymer. Specifically, first, a tantalum sintered body was immersed in the dispersion liquid of the second conductive polymer for about 3 to 10 seconds, and then the tantalum sintered body was pulled out of the dispersion liquid. Then, the tantalum sintered body pulled out of the dispersion liquid was heated at 180° C. for 20 minutes to form a second solid electrolyte layer. For the second conductive polymer, poly(3,4-ethylenedioxythiophene) (PEDOT) doped with polystyrene sulfonic acid (PSS) was used.

Next, a cathode extraction layer was formed on the second conductive polymer layer. In this manner, a capacitor element was formed. Then, lead terminals were connected to the capacitor element. Then, a portion of the lead terminals and the capacitor element were sealed with an exterior body. In this manner, the solid electrolytic capacitor (capacitor A1) was produced.

### (Capacitors A2 and A3)

Capacitors A2 and A3 were produced under the same conditions and using the same method as those of the capacitor A1, except that the formation method of the first solid electrolyte layer was changed. The first solid electrolyte layers in the capacitors A2 and A3 were formed under the same conditions and using the same method as those of the first solid electrolyte layer in the capacitor A1, except that the composition ratio of the monomers contained in the reaction solution was set to the ratio shown in Table 1.

### (Capacitors A4 to A8)

Capacitors A4 to A8 were produced under the same conditions and using the same method as those of the capacitor A1, except that the formation method of the first solid electrolyte layer was changed. The first solid electrolyte layers in the capacitors A4 to A8 were formed under the same conditions and using the same method as those of the first solid electrolyte layer in the capacitor A1, except that the above-described value X in the reaction solution was set to the value shown in Table 1.

### (Capacitor C1)

Capacitor C1 was produced under the same conditions and using the same method as those of the capacitor A1, except that the formation method of the first solid electrolyte layer was changed. The first solid electrolyte layer in the capacitor C1 was formed under the same conditions and using the same method as those of the first solid electrolyte layer in the capacitor A1, except that the monomer was 3,4-ethylenedioxythiophene (EDOT) alone.

### (Capacitor C2)

Capacitor C2 was produced under the same conditions and using the same method as those of the capacitor A1, except that the second solid electrolyte layer was not formed.

### (Capacitor C3)

Capacitor C3 was produced under the same conditions and using the same method as those of the capacitor A1, except that the formation method of the first solid electrolyte layer was changed. The first solid electrolyte layer in the capacitor C3 was formed under the same conditions and using the same method as those of the first solid electrolyte layer in the capacitor A1, except that a silane compound was not added to the reaction solution.

The capacitors obtained as described above were subjected to aging treatment for 90 minutes at 120° C., and 35 V. Next, the capacitors that had undergone the aging treatment were left at room temperature for 1 hour or more, and then the leakage current (LC) defect rate, the electrostatic capacitance (Cap), the equivalent series resistance (ESR), and the withstand voltage were measured using the methods described below.

### (Leakage Current Defect Rate)

In each of the 500 produced capacitors A1, a voltage of 35 V was applied between the anode lead terminal and the cathode lead terminal, and the leakage current (LC) after 120 seconds was measured. Capacitors with a leakage current exceeding 100 μA were determined as defective. The percentage of defective capacitors in the 500 capacitors A1 was determined as the LC defect rate (%). The LC defect rates of the other capacitors were also evaluated in the same manner.

### (Cap)

The electrostatic capacitances (Caps) of the capacitors A1 at a frequency of 120 Hz were measured using an LCR meter for four-terminal measurement. The Caps were measured for 10 capacitors A1 randomly selected from the capacitors A1 that were deemed good in the leakage current evaluation. The arithmetic average of the measured caps of the 10 capacitors was used for evaluation. The Caps of the other capacitors were also evaluated in the same manner.

### (ESR)

The ESR values (initial ESR values) of the capacitors A1 at a frequency of 100 kHz were measured using an LCR meter for four-terminal measurement. The ESRs were measured for 10 capacitors A1 randomly selected from the capacitors A1 that were deemed good in the leakage current evaluation. The arithmetic average of the ESR values measured for the 10 capacitors was used for evaluation. The ESRs of the other capacitors were evaluated in the same manner.

### (Withstand Voltage)

The withstand voltage of each capacitor was measured. Specifically, a voltage was applied to the capacitor while the voltage was raised at a rate of 1.0 V/sec, and the voltage at which an overcurrent of 0.5 A flowed was defined as withstand voltage.

Table 1 shows some of the manufacturing conditions and the evaluation results.

TABLE 1

| | Reaction solution | | | Second | Characteristics | | | |
|---|---|---|---|---|---|---|---|---|
| | Alkyl | | | solid | LC | | | Withstand |
| | EDOT/EDOT | Value | Value | electrolyte | defect | Cap | ESR | voltage |
| | (mole ratio) | Y | X | layer | rate (%) | (μF) | (mΩ) | (V) |
| A1 | 50/50 | 0.50 | 0.10 | Present | 1.2 | 42 | 45 | 61 |
| A2 | 75/25 | 0.75 | 0.10 | Present | 0.6 | 42 | 48 | 63 |
| A3 | 25/75 | 0.25 | 0.10 | Present | 6.0 | 42 | 44 | 56 |
| A4 | 50/50 | 0.50 | 0.05 | Present | 1.8 | 42 | 44 | 59 |
| A5 | 50/50 | 0.50 | 0.15 | Present | 0.4 | 42 | 47 | 63 |
| A6 | 50/50 | 0.50 | 0.20 | Present | 0.0 | 42 | 49 | 65 |
| A7 | 50/50 | 0.50 | 0.30 | Present | 0.0 | 41 | 62 | 69 |
| A8 | 50/50 | 0.50 | 0.40 | Present | 0.0 | 41 | 96 | 72 |
| C1 | 0/100 | 0.00 | 0.10 | Present | 18.8 | 41 | 46 | 52 |
| C2 | 50/50 | 0.50 | 0.10 | Absent | 32.2 | 41 | 58 | 40 |
| C3 | 50/50 | 0.50 | 0.00 | Present | 10.0 | 42 | 40 | 48 |

As shown in Table 1, the capacitors A1 to A8 had significantly lower LC defect rates and higher withstand voltages than the comparative capacitors C1 to C3. The capacitors A1 to A7 in which the values X were 0.30 or less had low ESRs. The capacitor C1 that did not use an alkyl EDOT, the capacitor C2 that did not include a second solid electrolyte layer, and the capacitor C3 in which a silane compound was not added to the reaction solution had significantly higher LC defect rates and lower withstand voltages. The capacitors A6 to A8 in which the values X were within a range of 0.20 to 0.40 had particularly low LC defect rates.

### INDUSTRIAL APPLICABILITY

The present disclosure can be used for a solid electrolytic capacitor and a manufacturing method therefor.

Although the present invention has been described with respect to the presently preferred embodiments, such disclosure should not be interpreted as being limiting. Various variations and modifications will no doubt become apparent to those skilled in the art to which the present invention pertains upon reading the above disclosure. Accordingly, the appended claims should be interpreted to encompass all variations and modifications without departing from the true spirit and scope of the present invention.

### REFERENCE SIGNS LIST

100: Electrolytic capacitor, 100: capacitor, 113: anode body, 113a: porous part, 114: dielectric layer, 116: solid electrolyte layer, 116a: first solid electrolyte layer, 116b: second solid electrolyte layer

1. A manufacturing method for a solid electrolytic capacitor that includes an anode body having a porous part on a surface thereof and a dielectric layer formed on at least a portion of a surface of the porous part, the manufacturing method comprising:
a step (i) of forming a first solid electrolyte layer that covers at least a portion of the dielectric layer; and
a step (ii) of forming a second solid electrolyte layer that covers at least a portion of the first solid electrolyte layer,
wherein the first solid electrolyte layer contains a first conductive polymer,
the second solid electrolyte layer contains a second conductive polymer,
the step (i) includes:
a step (i-a) of supplying a reaction solution containing a monomer and a silane compound to a surface of the dielectric layer; and
a step (i-b) of forming the first solid electrolyte layer by polymerizing the monomer in the supplied reaction solution to form the first conductive polymer, and
the monomer contains a compound represented by the following formula (I): 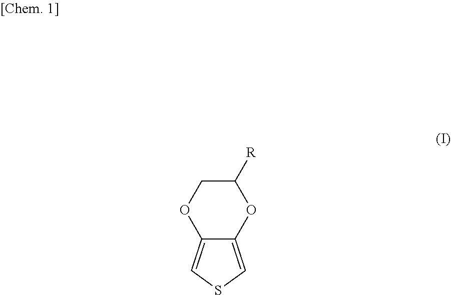
(in the formula (I), R represents an alkyl group whose carbon number is within a range of 1 to 10).

2. The manufacturing method according to claim 1, wherein the monomer further contains 3,4-ethylenedioxythiophene.

3. The manufacturing method according to claim 1,
wherein the silane compound is a silane coupling agent,
the reaction solution contains an oxidizing agent, and
in the reaction solution, a value of (mass of the silane compound)/(sum total of mass of the monomer, mass of the oxidizing agent, and mass of a liquid medium of the reaction solution) is within a range of 0.05 to 0.40.

4. The manufacturing method according to claim 1,
wherein the step (ii) includes:
a step (ii-a) of applying a dispersion liquid that contains the second conductive polymer and a dispersion medium to the first solid electrolyte layer; and
a step (ii-b) of forming the second solid electrolyte layer by removing at least a portion of the dispersion medium from the applied dispersion liquid.

5. The manufacturing method according to claim 1, wherein the anode body is a tantalum sintered body.

6. The manufacturing method according to claim 1, wherein the second conductive polymer contains poly(3,4-ethylenedioxythiophene).

7. A solid electrolytic capacitor comprising:
an anode body that has a porous part on a surface thereof;
a dielectric layer that is formed on at least a surface of a portion of the porous part;
a first solid electrolyte layer that covers at least a portion of the dielectric layer; and
a second solid electrolyte layer that covers at least a portion of the first solid electrolyte layer,
wherein the first solid electrolyte layer includes a first conductive polymer and a silicon-containing component,
the second solid electrolyte layer contains a second conductive polymer,
the first conductive polymer is a polymer of a monomer, and
the monomer contains a compound represented by the following formula (I): 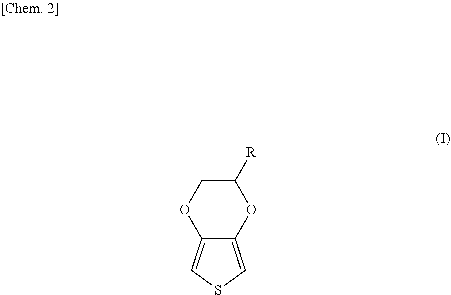
(in the formula (I), R represents an alkyl group whose carbon number is within a range of 1 to 10).

8. The solid electrolytic capacitor according to claim 7, wherein the monomer further contains 3,4-ethylenedioxythiophene.

9. The solid electrolytic capacitor according to claim 7, wherein the silicon-containing component is derived from a silane coupling agent.

10. The solid electrolytic capacitor according to claim 7, wherein the anode body is a tantalum sintered body.

11. The solid electrolytic capacitor according to claim 7, wherein the second conductive polymer contains poly(3,4-ethylenedioxythiophene).
